(19) United States
(12) Patent Application Publication
(10) Pub. No.: US 2011/0044947 A1
(43) Pub. Date: Feb. 24, 2011

(54) METHODS OF TREATING CARDIAC DISORDERS

(76) Inventors: Victor Dzau, Newton, MA (US); Luis G. Melo, Saskatoon (CA); Mark A. Perrella, Boston, MA (US); Reitu Agrawal, Coraville, IA (US)

Correspondence Address: MINTZ, LEVIN, COHN, FERRIS, GLOVSKY AND POPEO, P.C, ONE FINANCIAL CENTER, BOSTON, MA, 02111, US

(21) Appl. No.: 12/731,550

(22) Filed: Mar. 25, 2010

Related U.S. Application Data
(63) Continuation of application No. 11/603,271, filed on Nov. 20, 2006, now abandoned.

(51) Int. Cl. A61K 48/00; C12N 15/85; C12N 15/86; C12N 15/861; A61P 9/00; C12N 15/867; C12N 15/53

(52) U.S. Cl. 424/93.2; 536/23.2; 435/320.1; 514/44 R

(57) ABSTRACT

The invention feature methods and compositions for treating ischemic and reperfusuin related injury such as cardiac disorders.

### RELATED APPLICATIONS

This application is a continuation of U.S. patent application Ser. No. 11/603,271, filed Nov. 20, 2006, which claims the benefit of U.S. patent application Ser. No. 10/161,921, filed Jun. 3, 2002, which claims the benefit of U.S. Provisional Application No. 60/295,229, filed Jun. 1, 2001, the contents of each of which are incorporated by reference in its entirety.

### STATEMENT AS TO FEDERALLY SPONSORED RESEARCH

This invention was made with U.S. government support under National Institutes of Health grants. The government has certain rights in the invention.

### FIELD OF THE INVENTION

The invention relates to methods of treating cardiac disorders.

### BACKGROUND OF THE INVENTION

Chronic coronary artery disease is a leading cause of death in the Western world. Depending upon their duration and severity, single or intermittent ischemic episodes may leads to contractile dysfunction, myocardial cell injury and death. Although timely re-establishment of blood flow to an ischemic area is an obligatory requirement for myocardial salvage, there is evidence that reperfusion subsequent to ischemia triggers a series of cytotoxic and inflammatory events that can exacerbate irreversible tissue damage initiated during ischemia. Several lines of evidence suggest that a significant component of cellular injury associated with reperfusion is caused by a burst in the generation of highly reactive free radical oxygenase species (ROS), upon reoxygenation of the myocardium and subsequent activation of the inflammatory cascade. The accumulation of ROS may eventually deplete the buffering capabilities of endogenous antioxidant systems, thereby exacerbating the deleterious effects of these reactive species.

### SUMMARY OF THE INVENTION

The invention features methods of inhibiting cell or tissue damage, e.g., cardiomyocyte cell death or inhibiting an ischemic or reperfusion related injury. Ischemia causes irreversible cellular/tissue damage and cell death. Reperfusion exacerbates ischemic damage by activating inflammatory response and oxidative stress. Oxidative stress modifies membrane lipids, proteins and nucleic acids resulting in cellular/tissue damage or death, and depression of cardiac, endothelial and kidney function.

Cell damage or injury is inhibited in a subject by administering to the subject prior to identification of the cell damage or injury a composition containing a nucleic acid encoding a cell protective polypeptide or a biologically active fragment thereof where the expression of the polypeptide is induced by a triggering agent or condition.

The invention also features methods of preventing cardiomyocyte death in a subject (e.g., mammal) by administering to the subject a composition containing a nucleic acid encoding a human heme oxygenase-1 polypeptide or extracellular superoxide dismutase (ecSOD) polypeptide or a biologically active fragment thereof. The subject is suffering from or at risk of developing a cardiac disorder such as a chronic cardiac disorder.

Also featured by the invention is a composition containing a nucleic acid encoding a cell protective polypeptide, one or more oxygen sensitive regulatory elements which regulates the expression of the polypeptide and a cell targeting element. Alternatively, the composition contains two, three, five, seven or ten oxygen sensitive regulatory elements.

Preferably, the composition is administered prior to an ischemic event such as a cardiac event, e.g., a myocardial infarction, stroke, hypertension, congestive heart failure, dilated cardiomyopathy, or restenosis. Alternatively, the composition is administered after a the event, e.g., 2 months, three months, six months or one year or two year, to prevent potential myocardial injury from a future cardiac event.

The subject may be suffering from or at risk of developing a condition characterized by aberrant cell damage such as oxidative-stress induced cell death (e.g., apoptotic cell death) or an ischemic or reperfusion related injury. A subject suffering from or at risk of developing a condition is identified by the detection of a known risk factor, e.g., gender, age, high blood pressure, obesity, diabetes, prior history of smoking, stress, genetic or familial predisposition, attributed to the particular disorder, or previous cardiac event such as myocardial infarction or stroke.

Conditions characterized by aberrant cell death include cardiac disorders (acute or chronic) such as stroke, myocardial infarction, chronic coronary ischemia, arteriosclerosis, congestive heart failure, dilated cardiomyopathy, restenosis, coronary artery disease, heart failure, arrhythmia, angina, atherosclerosis, hypertension, renal failure, kidney ischemia or myocardial hypertrophy.

A cell protective polypeptide is a polypeptide which is capable of inhibiting cell damage such as oxidative-stress induced cell death. Suitable tissue protective polypeptides include for example an antioxidant enzyme protein, a heat shock protein, an anti-inflammatory protein, a survival protein, a -pro-apoptotic protein, a coronary vessel tone protein, a pro-angiogenic protein, a contractility protein, a plaque stabilization protein, a thromboprotection protein, a blood pressure protein and a vascular cell proliferation protein. Preferably the cell protective polypeptide is a human heme oxygenase-1 polypeptide or a human extracellular superoxide dismutase or a biologically active fragment thereof. Exemplary human heme oxygenase-1 polypeptides includes for example GenBank Accession numbers P09601 and CAA32886. Exemplary human extracellular superoxide dismutase polypeptides includes for example GenBank Accession numbers Q07449 and P08294.

The triggering agent or condition is endogenous or exogenous. All that is required is that the agent or condition induces the expression of the cell protective polypeptide. Preferably, induction is temporal. Induction of expression of the polypeptide occurs either pre-translation (e.g., via enhancers, promoters, response elements such as hypoxia or antioxidant response elements) or post-translation. For example the condition is a physiological stimulus such as hypoxia, oxidative stress, reactive oxygen species such as hydrogen peroxide, superoxide or hydroxyl radicals. The agent is an antibiotic such as tetracycline; an immunosuppressive such as rapamycin; a steroid hormone such as ecdysone; or a hormone receptor antagonist such as mifepristone. Alternatively, the triggering agent is a member of a binary gene expression system such as the tetracycline responsive expression system or the ecdysone responsive expression system.

An oxygen sensitive regulatory element is an element that is modified by hypoxia or oxidative stress and is capable of regulating (e.g., turning on or turning off) expression of the cell protective polypeptide. For example, an oxygen sensitive regulatory element is a hypoxia-responsive element (HRE), antioxidant response elements (ARE) or an oxidative stress response element such as a peroxidase promoter or nuclear factor kappa B (NF-κB).

The cell targeting element is an element that is capable of restricting expression of the cell protective polypeptide to the cell type of interest, e.g., cardiac tissue or kidney tissue. For example a cell targeting element is a cell-specific promoter (e.g., α-MHC, myosin light chain-2, or troponin T).

To determine whether the composition inhibits oxidative-stress induced cell death, the composition is tested by incubating the composition with a primary or immortalized cell such as a cardiomyocyte. A state of oxidative stress of the cells is induced (e.g., by incubating them with H₂O₂) and cell viability is measured using standard methods. As a control, the cells are incubated in the absence of the composition and then a state of oxidative stress is induced. A decrease in cell death (or an increase in the number of viable cells) in the compound treated sample indicates that the composition inhibits oxidative-stress induced cell death. Alternatively, an increase in cell death (or an decrease in the number of viable cells) in the compound treated sample indicates that the composition does not inhibit oxidative-stress induced cell death. The test is repeated using different doses of the composition to determine the dose range in which the composition functions to inhibit oxidative-stress induced cell death.

The nucleic acid compositions are formulated in a vector. Vectors include for example, an adeno-associated virus vector, a lentivirus vector and a retrovirus vector. Preferably the vector is an adeno-associated virus vector. Preferably the nucleic acid is operatively linked to a promoter such as a human cytomegalovirus immediate early promoter. An expression control element such as a bovine growth hormone polyadenylation signal is operably-linked to coding region the cell protective polypeptide. In preferred embodiments, the nucleic acid of the is flanked by the adeno-associated viral inverted terminal repeats encoding the required replication and packaging signals.

The invention further features a method of treating a chronic cardiac disorder by identifying a mammal suffering from or at risk of developing a chronic cardiac disorder by administering to the mammal a composition including a nucleotide encoding a human heme oxygenase-1 polypeptide or a biologically active fragment thereof. A biologically active polypeptide of HO has an amino acid sequence less than that of a naturally occurring HO polypeptide and which inhibits oxidative stress-induced cardiomyocyte death. A chronic cardiac disorder includes disorders such as, chronic coronary ischemia, arteriosclerosis, congestive heart failure, angina, atherosclerosis, and myocardial hypertrophy.

The invention further features a method of treating a cardiac disorder subject a composition including a nucleotide encoding a extracellular superoxide dismutase (ecSOD) polypeptide or a biologically active fragment thereof. A biologically active polypeptide of ecSOD polypeptide has an amino acid sequence less than that of a naturally occurring ecSOD polypeptide and which inhibits oxidative stress-induced cardiomyocyte death. The subject can be at risk if a cardiac disorder such as myocardial infarction, chronic coronary ischemia, arteriosclerosis, congestive heart failure, angina, atherosclerosis, and myocardial hypertrophy.

Also provided by the invention, is a cardioprotective agent including a recombinant adeno-associated viral vector and a nucleotide encoding a human heme oxygenase-1 polypeptide or a human extracellular superoxide dismutase polypeptide operatively linked to a human cytomegalovirus immediate early promoter. Preferably, the cardioprotective agent includes a bovine growth hormone polyadenylation signal. More preferably, the bovine growth hormone polyadenylation signal is flanked by the adeno-associated viral inverted terminal repeats.

Other features and advantages of the invention will be apparent from the following description of the preferred embodiments thereof, and from the claims.

### BRIEF DESCRIPTION OF THE DRAWINGS

FIG. 1A is an illustration showing a viral vector expressing human heme oxygenase 1 (hHO-1) cDNA under transcriptional control of the human cytomegalovirus (CMV) early gene promoter, rAAV_{CMV-hHO-1}. ITR indicates the adeno-associated virus (AAV) inverted terminal repeats encoding replication and packaging signals. BGH-pA indicates the bovine growth hormone polyadenylation signal.

FIG. 1B is an illustration of a heart showing the approximate area at risk of infarction (delineated by dotted line, based on Evans Blue exclusion) after ligation of left anterior descending coronary artery and sites of injection of the rAAV_{CMV-hHO-1} viral vector (filled circles)

FIG. 1C is a photograph showing an electrophoretic gel showing hHO-1 gene expression in left ventricle eight weeks after rAAV-mediated intramyocardial gene transfer as measured by reverse transcriptase-polymerase chain reaction (RT-PCR). Lanes 1 and 2, rAAV_{CMV-hHO-1}-transduced left ventricle myocardium in two separate animals. Lane 3, rAAV_{CMV-LacZ}-transduced heart. Lane 4, untransduced heart. Lane 5, hHO-1 cDNA (positive control).

FIG. 1D is a photograph of a Western Blot of proteins derived from hHO-1 transduced cells showing the distribution and abundance of HO-1 protein expression from apical (lane 1), mid (lane 2) and basal (lane 3) regions.

FIG. 1E is a photograph of a Western Blot of proteins derived from hHO-1 control (untransduced) cells showing the distribution and abundance of 140-1 protein expression from apical (lane 1), mid (lane 2) and basal (lane 3) regions.

FIG. 1F is a photomicrograph of cardiac tissue showing localization of HO-1 protein in paraffin-embedded cross sections from rAAV_{CMV-hHO-1} transduced hearts by immunohistochemical analysis.

FIG. 1G is a photomicrograph of cardiac tissue showing localization of HO-1 protein in paraffin-embedded cross sections from control (untransduced) hearts by immunohistochemical analysis.

FIG. 2A is a bar graph showing planar morphometric analysis of area at risk in hHO-1-transduced (HO-1, n=6) LacZ-transduced (LacZ, n=5) and saline-injected (saline, n=6) control hearts. Values are means±SE.

FIG. 2B is a bar graph showing planar morphometric analysis of infarct area in hHO-1-transduced (HO-1, n=6) LacZ-transduced (LacZ, n=5) and saline-injected (saline, n=6) control hearts. Values are means±SE.

FIG. 2C is a bar graph showing planar morphometric analysis of infarct size in hHO-1-transduced (HO-1, n=6) LacZ-transduced (LacZ, n=5) and saline-injected (saline, n=6) control hearts. Values are means±SE.

FIG. 3A is a bar graph showing total basal heme oxygenase activity in left ventricle microsomal extracts after 30 min of ischemia and 24 hr of reperfusion. (*, P<0.05).

FIG. 3B is a photograph of a Western Blot showing HO-1 protein expression in homogenates from hHO-1-transduced (lane 1), LacZ-transduced (lane 2), saline injected (lane 3) and sham operated (lane 3) hearts.

FIG. 3C is a photograph of a Western Blot showing HO-2 protein expression in homogenates from hHO-1-transduced (lane 1), LacZ-transduced (lane 2), saline injected (lane 3) and sham operated (lane 3) hearts.

FIG. 4A is a bar graph showing oxidative stress-induced lipid peroxide production from hHO-1-transduced (open bar), LacZ-transduced (closed bar), saline injected (hatched bar) and sham operated (stippled bar) hearts. (*, P<0.05).

FIGS. 4B-F are photographs of a Western Blot assay showing the expression of apoptosis-related proteins and inflammatory cytokines in left ventricle homogenates after 30 min of ischemia and 24 hr of reperfusion. Lane 1 shows proteins derived from transduced heart tissue, lane 2 shows proteins derived from untransduced heart tissue, and lane 3 shows proteins derived from sham-operated heart tissue. FIG. 4B shows BAX expression. FIG. 4B shows Bcl2 expression. FIG. 4B shows IL-1β expression. FIG. 4B shows TNFα expression.

FIG. 5A is a photograph of a Western Blot assay showing the expression of HO-1 expression in transgenic mice.

FIG. 5B is a bar graph showing the size of infarct is greater in wild-type mice (open bar) compared to transgenic mice which overexpress HO-1 (closed bar).

FIG. 6A is an illustration showing an rAAV-ecSOD vector expressing human SOD under transcriptional control of the CMV promoter. ITR indicates the AAV inverted repeats and pA the bovine growth hormone polyadenylation signal

FIG. 6B is an illustration showing RT-PCR detection of ecSOD transcript in the left ventricle 8 weeks after rAAV mediated gene transfer. Lane M: marker; Lanes 1-3: rAAV-ecSOD transduced hearts; Lanes 4-6:rAAV-LAcZ transduced hearts; lane 7: human ecSOD cDNA (positive control); Lane 8: untransduced heart (negative control).

FIG. 6C is an illustration of a Western blot assay showing the expression of ecSOD protein form control; and three ecSOD transduced hearts.

FIG. 7A is a bar graph showing planer morphometric analysis of serial heart sections showing areas of risk.

FIG. 7B is a bar graph showing planer morphometric analysis of serial heart sections showing areas that the infarct size (% of area at risk) in hEC-SOD was reduced compared to control animal receiving LacZ reporter gene.

FIG. 8 is a illustration showing the effect of intramyocardial rAAV-hSOD gene transfer on long term animal survival compared to LacZ.

FIG. 9 is an illustration showing the construction of the pGL3 EpoHRE vector.

FIG. 10A is bar graphs showing the effect of hypoxia on HRE mediated induction of luciferase activity of 293 cells transfected with a vector containing fCMV promoter alone or 4EpoHRE-mCMV vector. Open bars normoxic condition, closed bars hypoxic conditions.

FIG. 10B is bar graphs showing the effect of hypoxia on HRE mediated induction of luciferase activity of 293 cells transfected with a vector containing fCMV promoter alone, 4EpoHRE-mCMV or 3EpoHRE-fCMV vectors. Open bars normoxic condition, closed bars hypoxic conditions.

FIG. 11A is a photograph showing the gross histological appearance on infarct in TTC-stained sections of rAAV-hHO-1 treated animals one month after acute myocardial infarction. FIG. 11B are bar graphs showing planar morphometry of biventricular thick sections f rAAV-hHO-1 treated animals one month after acute myocardial infarction. Infarct size was reduced by 90% in the HO-1 treated group (n=4) relative to the LacZ treated animals (n=7).

FIG. 12 is a photograph of RT-PCR detection of LacZ transcripts in extracardiac tissues 6 months after intermyocardial injection of rAAV-LacZ.

### DETAILED DESCRIPTION

Advances in the understanding of the molecular mechanisms of heart disease and in the development of efficient gene delivery systems offered the opportunity to design gene-based therapies for myocardial protection. The present invention provides compositions and methods for a preventive gene therapy strategy for myocardium protection from future ischemia/reperfusion (I/R) injury involving a single administration of a therapeutic gene with a vector system capable of efficient and long-term myocyte specific and inducible expression of the therapeutic gene. Using a vector system (e.g., recombinant adenoassociated viral vectors (rAAV)) which allows for long-term and stable expression of transduced genes in the myocardium, together with cis-acting promoter elements that are capable of conferring an inducible and cell-specific gene expression, temporal and spatial control of expression of the therapeutic transgene is achieved. The results demonstrate that that transgenic mice with cardiac-directed overexpression of HO-1 or ecSOD develop resistance to I/R-induced myocardial injury. Furthermore, a single intramyocardial delivery of HO-1 gene or ecSOD gene by rAAV in rats, eight weeks in advance of I/R-induced myocardial injury, resulted in dramatic reduction in myocardial infarction.

### Coronary Disorders

Coronary disorders, can be categorized into at least two groups. Acute coronary disorders include myocardial infarction, and chronic coronary disorders include chronic coronary ischemia, arteriosclerosis, congestive heart failure, angina, atherosclerosis, and myocardial hypertrophy. Other coronary disorders include stroke, myocardial infarction, dilated cardiomyopathy, restenosis, coronary artery disease, heart failure, arrhythmia, angina, or hypertension.

Acute coronary disorders result in a sudden blockage of the blood supply to the heart which deprives the heart tissue of oxygen and nutrients, resulting in damage and death of the cardiac tissue. In contrast, chronic coronary disorders are characterized by a gradual decrease of oxygen and blood supply to the heart tissue overtime causing progressive damage and the eventual death of cardiac tissue.

### Tissue Protective Polypeptides

Table I provides a list of tissue protective polypeptides useful in the compositions and methods of the invention.

TABLE I
Targets for gene-based therapy for congenital and acquired heart disease.

| Strategy | Therapeutic Target | Genetic manipulation | Vector | Application |
|---|---|---|---|---|
| Protection/Prevention | | | | |
| Antioxidant enzymes | HO-1, SOD, catalase, GPx | overexpression | AAV, LV | CAD, MI |
| Heat shock proteins | HSP70, HSP90, HSP27 | overexpression | AAV, LV | CAD, MI |
| Anti-inflammatory | I-CAM, V-CAM, NF-κB, TNF-α | inhibition | AS-ODN | graft atherosclerosis |
| | | | Decoy ODN | transplantation |
| | | | AAV-AS-ODN | |
| | | | RV-AS-ODN | |
| Survival genes | Bcl-2, Akt | overexpression | AAV, LV | CAD, MI, HF |
| Pro-apoptotic genes | Bad, p53, Fas ligand | inhibition | AS-ODN | MI, HF |
| | | | Decoy ODN | |
| | | | AAV-AS-ODN | |
| Coronary vessel tone | eNOS, adenosine (P1, P3) receptors | overexpression | RV, AAV | CAD, HF |
| Rescue | | | | |
| Pro-angiogenic genes | VEGF, FGF, HGF | overexpression | AAV | CAD, MI, HF |
| Contractility | β-adrenergic receptors, | overexpression | AAV | HF |
| | SERCA 2A, V1 receptor | | | |
| | BARK, Phosphalamban | Inhibition | AAV | HF |
| Plaque stabilization | CD40 | overexpression | RV, AAV (?) | CAD |
| Thromboprotection | PAI-1, plasminogen activator | inhibition | AS-ODN | CAD, MI |
| | Tissue factor | | | |
| | TPA, hirudin, urokinase | overexpression | AAV | CAD, MI |
| | Thrombomodulin, COX-1, | | | |
| | PGI₂ synthase | | | |
| Blood pressure | Kallikrein, eNOS, ANP | overexpression | AAV, RV | hypertension, HF |
| | ACE, AGT, AT₁ | inhibition | AAV-AS-ODN | |
| Vascular cell proliferation | NOS, Ras dominant negative | overexpression | AD, RV, AAV | graft atherosclerosis |
| | E2F, c-myb, c-myc, PCNA | inhibition | AS-ODN, | restenosis |
| | | | Decoy-ODN | |
| Inherited heart disease | | | | |
| Channelopathies | SCN5A, Iₖ | overexpression | α-MHC-AAV | arrhythmia |
| Cardiomyopathy | sarcomeric proteins, sarcoglycans | overexpression | α-MHC-AAV | DCM |
| | | | | (in utero) |

Abbreviations:
AAV, adeno-associated virus;
AS-ODN, antisense oligodeoxynucleotide;
CAD, coronary artery disease;
DCM, dilated cardiomyopathy;
HF, heart failure;
LV, lentivirus;
MI, myocardial infarctionα-MHC, alpha myosin heavy chain;
RV, retrovirus,
HO-1, Heme oxygenase-1;
SOD, superoxide dismutase;
GPx, glutathione peroxidase;
HSP70, 70 kD heat shock protein;
HSP90, 90 kD heat shock protein;
HSP27, 27 kD heat shock protein;
I-CAM, intercellular adhesion molecule;
V-CAM, vascular adhesion molecule;
NF-κB, nuclear factor kappa B;
TNF-α, tumor necrosis factor alpha;
eNOS, endothelial nitric oxide synthase;
VEGF, vascular endothelial growth factor;
FGF, fibroblast growth factor;
HGF, hematopoietic growth factor,
SERCA 2A, sarcoplasmic/endoplasmic reticulum Ca-2+ ATPase;
V1 receptor, vasopressin-1 receptor;
bARK, beta-adrenergic receptor kinase;
PAI-1, plasminogen activator inhibitor-1;
TPA, tissue plasminogen activator;
COX-1, cyclooxygenase-1;
PGI₂ synthase, prostacyclin synthase;
ANP, atrial natriuretic peptide;
ACE, angiotensin-converting enzyme;
AGT, angiotensinogen;
AT₁, Angiotensin II-type-1;
NOS, nitric oxide synthase;
PCNA, proliferating cell nuclear antigen;
SCN5A, cardiac sodium channel gene 5A.

### Regulatable Gene Expression

Effective gene therapy requires that gene expression is regulated in order to achieve optimal expression levels and reduce side effects associated with constitutive gene expression. An ideal strategy for myocardial protection against ischemia/reperfusion injury with minimal potential side effects resulting from constitutive expression of the transgene is a regulatable expression system. It would be desirable to turn on gene expression with the onset of ischemia (hypoxia), so that the gene product would be already present during reperfusion.

Many transcription factors are modified by hypoxic and oxidative stress, studies of molecular responses to hypoxia have identified HIF-I as the master regulator of hypoxia-inducible gene expression. Under hypoxic conditions, HIF-I binds to the hypoxia-responsive element (HRE) in the enhancer region of its target genes and turns on gene transcription. Additionally, reperfusion or reoxygenation after ischemia increases the transactivating ability of NF_{K}B Genes regulated by NF_{K}B include cytokines and adhesion molecules, which by promoting inflammatory responses, contribute to cell death. Several studies indicate that the hypoxic and hyperoxic environment can be used to activate heterologous gene expression driven by HRE and cis-acting consensus sequences of activated NF_{K}B respectively. Accordingly, in one aspect of the invention HRE are utilized as an enhancer to drive transgene expression. To assure sufficient duration of the transgene expression to achieve myocardial protection during the reperfusion period, as second regulatory element that is activated by oxidative stress such as NF_{K}B responsive element can be utilized.

### Cell Specific Gene Expression

The potential applications of gene therapy are currently limited by the absence of efficient cell-specific targeting vectors. This lack of tissue specificity is a fundamental problem for gene therapy as proteins that are therapeutic in target cells also may be harmful to normal tissue. Thus non cell-specific expression of a transgene has the potential for inducing metabolic and physiologic mechanisms that could result in pathology over the long term. Localized injections can provide certain degree of localized expression of the targeting vector, however, there may still be a spill over into the circulation which will affect other cells and organs. One way to circumvent this problem is to use transcriptionally targeted vectors that can restrict the expression of the therapeutic proteins primarily to the target cells by the use of tissue-specific promoters (e.g. a-myosin heavy chain, myosin light chain. The cells in the myocardium that are particularly prone to reperfusion injury are the cardiac myocytes and microvascular endothelial cells. Thus, a cell specific strategy could be directed to protect either cell type.

Myocardial Protection with HO-1 and ecSOD Gene Expression

Recombinant adeno-associated virus (rAVV) for direct delivery of the human heme oxygenase gene (hHO-1) or extracellular superoxide dismutase (ecSOD) into the rat myocardium, was evaluated as a strategy for a therapeutic approach for the long term protection from oxidative stress-mediate myocardial injury.

The selection of HO-1 as a therapeutic agent was made on the basis of evidence that the enzyme neutralizes the potent pro-oxidant activity of heme and that its multiple catalytic by-products bilirubin, carbon monoxide (CO) and free iron together exert powerful, pleiotropic cytoprotective effects. Bilirubin is a potent endogenous antioxidant that scavenges peroxyl radicals and reduces peroxidation of membrane lipids and proteins. CO is a vasodilator and powerful anti-inflammatory and antiapoptotic agent. Free iron stimulates the synthesis of the iron binding protein ferritin, which reduces iron-mediated formation of free radicals and upregulates several key cytoprotective genes.

A recognized coronary artery ligation and release model of oxidative injury was used to determine whether (1) rAAV is an efficient vector for in vivo transfer of therapeutic genes into the myocardium, (2) rAAV provides stable and sustained expression of the transgene, and (3) HO-1 and or ecSOD as suitable cytoprotective genes for oxidative stress induced myocardial injury. The findings indicate that intramyocardial delivery of a rAAV-hHO-1 gene construct results in prolonged expression of the transgene and leads to almost complete obliteration of myocardial infarction following 30 min/24 hour of ischemia/reperfusion eight weeks after gene transfer. Similarly, the finding were obtained for the that rAAV-ecSOD gene construct. These findings indicate that gene therapy using a rAAV vector/HO-1 gene or rAAV vector/ecSOD gene combination is efficacious for long-term protection from myocardial ischemia/reperfusion injury. These constructs are useful for a preventative and or adjunct therapy for chronic myocardial ischemic and inflammatory disease.

### Gene Therapy to Cardiac Tissue

Gene therapy refers to therapy that is performed by the administration of a specific nucleic acid to a subject. A nucleic acid is delivered to a target cell which in turn produces a gene product that exerts a therapeutic effect, e.g., inhibition of cell damage such as cardiomyocyte death due to a pathological chronic cardiac disorder. Standard gene therapy methods known in the art may be used in the practice of the present invention. See e.g., Goldspiel, et al., 1993. Clin Pharm 12: 488-505.

In one aspect of the invention a nucleic acid or nucleic acids encoding a cell protective polypeptide such as for example human heme oxygenase-1 polypeptide (hHO-1), or functional derivatives thereof, or human extracellular superoxide dismutase (ecSOD) are administered by way of gene therapy. A therapeutic regimen is carried out by identifying a mammal, e.g., a human patient suffering from (or at risk of developing) a cardiac disorder, (i.e., chronic or acute) or ischemic or reperfusion related injury using standard methods. Alternatively, a therapeutic regimen is carried out prophylactically, particularly after the detection known risk factor, e.g., genetic or familial predisposition, attributed to the particular disease or previous cardiac or ischemic event.

A therapeutic composition contains an cell protective polypeptide nucleic acid in an expression vector. One type of vector is a “plasmid”, which refers to a linear or circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. Suitable expression vectors, include viral vectors (e.g., replication defective retroviruses, adenoviruses and adeno-associated viruses). Additionally, some viral vectors are capable of targeting a particular cells type either specifically or non-specifically.

The recombinant expression vectors contain a nucleic acid in a form suitable for expression in a target cell, e.g., myocardium cell. Recombinant expression vectors include one or more regulatory sequences, operatively linked to the nucleic acid sequence to be expressed. For example, the vector includes a promoter and/or an enhancer sequence which preferentially directs expression of a nucleic acid in vascular, e.g., cardiac-restricted ankyrin repeat protein promoter. Operably linked is means that the nucleotide sequence of interest is linked to the regulatory sequence(s) in a manner that allows for expression of the nucleotide sequence (e.g., in an in vitro transcription/translation system or in a host cell when the vector is introduced into the host cell). The term “regulatory sequence” includes promoters, enhancers and other expression control elements (e.g., polyadenylation signals). Such regulatory sequences are described, for example, in Goeddel; GENE EXPRESSION TECHNOLOGY: METHODS IN ENZYMOLOGY 185, Academic Press, San Diego, Calif. (1990).

The promoter may be inducible or constitutive, and, optionally, tissue-specific. The promoter may be, e.g., viral or mammalian in origin. Preferably the promoter is a human cytomegalovirus immediate early promoter. A nucleic acid molecule composition contains an expression control element that is operably-linked to coding region(s) of a cell protective polypeptide (e.g., hHO-1 polypeptide or an ecSOD polypeptide). Preferably, the expression control element is a bovine growth hormone polyadenylation signal. A polypeptide encoding a nucleic acid molecule and regulatory sequences are flanked by regions that promote homologous recombination at a desired site within the genome, thus providing for intra-chromosomal expression of nucleic acids. For example, the nucleic acid molecule is flanked by the adeno-associated viral inverted terminal repeats encoding the required replication and packaging signals. See e.g., Koller and Smithies, 1989. Proc Natl Acad Sci USA 86: 8932-8935. Alternatively, the nucleic acid that remains episomal and induces an endogenous gene, e.g., an endogenous HO gene.

Delivery of the therapeutic nucleic acid into a patient may be either direct (i.e., a nucleic acid or nucleic acid-containing vector is administered in vivo to patient tissues) or indirect (i.e., autologous or heterologous cells are contacted with the nucleic acid in vitro, then transplanted into the patient). These two approaches are known, respectively, as in vivo or ex vivo gene therapy. The nucleic acid is delivered to a target cell in the same manner such that it becomes intracellular (e.g., by infection using a defective or attenuated retroviral or other viral vector; see U.S. Pat. No. 4,980,286); directly injecting naked DNA; using microparticle bombardment (e.g., a “Gene Gun®; Biolistic, DuPont); coating the nucleic acids with lipids; co-administering a cell-surface receptors/transfecting agents; encapsulating the nucleic acid in liposomes, microparticles, or microcapsules; linking the composition to a peptide that is known to enter the nucleus. Nucleic acid compositions are associated with a ligand which facilitates receptor-mediated endocytosis (see, e.g., Wu and Wu, 1987. J Biol Chem 262: 4429-4432), to “target” cell types that specifically express the receptors of the linked ligand.

In Vivo Gene Therapy

Cell protective polypeptide constructs are prepared for therapeutic use, using known methods. The viral stock can be in the form of an injectable preparation containing pharmaceutically acceptable carrier such as saline. The final titer of the vector in the injectable preparation is preferably in the range of 10⁷-10¹³ viral particles. Useful concentrations for other gene therapy agents as well as other therapeutic and diagnostic agents will vary considerably, however suitable concentration for the particular agent can be determined by one of skill in the art.

The cell protective polypeptide constructs are delivered to the myocardium by direct intracoronary injection through the chest wall or using standard percutaneous catheter based methods under fluoroscopic guidance. The nucleic acids are administered at an amount sufficient for the transgene to be expressed to a degree which allows for highly effective therapy. For example, 4×10¹⁰-4×10¹² particles per 750 microliters. The injection is made in situ to the ventricular wall. Any variety of coronary catheter, or a perfusion catheter, is used to administer the nucleic acid solution. In addition, other techniques known to those having ordinary skill in the art can be used for transfer of genes to the myocardium. For example, a nucleic acid coated or impregnated stent is placed in a coronary vessel.

The agent may be delivered in a form that keeps the nucleic acid associated with the target tissue for an extended period of time, such as with a viscosity-enhancing to produce a thixotropic gel. For example, poloxamer 407 combined with a viral vector is useful to achieve gene transfer in vascular smooth muscle cells. March K L et al., “Pharmacokinetics of Adenoviral Vector-Mediated Gene Delivery to Vascular Smooth Muscle Cells: Modulation by Poloxamer 407 and Implications for Cardiovascular Gene Therapy,” Human Gene Therapy 6:41-53 (1995).

A biocompatible polymer matrix, e.g., a hydrogel, is used as an excipient. Suitable polymeric materials may comprise polyurethane, polydimethylsiloxane, ethylene vinyl acetate, polymethyl methacrylate, polyamide, polycarbonate, polyester, polyethylene, polypropylene, polystyrene, polyvinyl chloride, polytetrafluoroethylene and cellulose acetate or a mixture of the above or copolymers. These non-biodegradable polymers may be employed as hollow reservoirs or as a structure for an implantable devise, which slowly administers the nucleic acid to surrounding tissue.

Indirect Gene Therapy

An indirect approach to gene therapy involves transferring a gene into cells in in vitro tissue culture by such methods as electroporation, lipofection, calcium phosphate-mediated transfection, viral infection, or the like. Generally, the methodology of transfer includes the concomitant transfer of a selectable marker to the cells. The cells are then placed under selection pressure (e.g., antibiotic resistance) so as to facilitate the isolation of those cells that have taken up, and are expressing, the transferred gene. Prior to the in vivo administration of the resulting recombinant cell, the nucleic acid is introduced into a cell by any method known within the art including, but not limited to transfection, electroporation, microinjection, infection with a viral or bacteriophage vector containing the nucleic acid sequences of interest, cell fusion, chromosome-mediated gene transfer, microcell-mediated gene transfer, spheroplast fusion, and similar methodologies that ensure that the necessary developmental and physiological functions of the recipient cells are not disrupted by the transfer. See e.g., Loeffler and Behr, 1993. Meth Enzymol 217: 599-618. The gene transfer method lead to stable transfer of the nucleic acid to the cell; the transferred nucleic acid is heritable and expressible by the cell progeny. Those cells are then delivered to a patient.

The resulting recombinant cells are delivered to a patient by various methods known within the art including, but not limited to, injection of transfected cells (e.g., subcutaneously) or directly into cardiac tissue. For example, HO nucleic acid constructs are introduced into autologous or histocompatible epithelial cells and recombinant skin cells are applied as a skin graft onto the patient.

The total amount of cells that are envisioned for use depend upon the desired effect, patient state, and the like, and may be determined by one skilled within the art. Dosages for any one patient depends upon many factors, including the patient's size, body surface area, age, the particular compound to be administered, sex, time and route of administration, general health, and other drugs being administered concurrently.

Cells into which a nucleic acid can be introduced for purposes of gene therapy encompass any desired, available cell type, and may be xenogeneic, heterogeneic, syngeneic, or autogeneic. Cell types include, but are not limited to, differentiated cells such as epithelial cells, endothelial cells, cardiomyoctes, fibroblasts, muscle cells, or various stem or progenitor cells, in particular embryonic heart muscle cells, liver stem cells (International Patent Publication WO 94/08598), and the like. Preferably the cells utilized for gene therapy are autologous to the patient.

### Example 1

### General Methods

The data described herein was generated using the following reagents and methods.

Plasmids and hHO-1 Vector Construction

A 986 by fragment of hHO-1 containing the open reading frame sequence was cleaved from the pBS KS (−) cloning vector at KpnI-PstI sites and subcloned at the corresponding sites in puc 18 plasmid. The insert was cut at EcoRI sites and cloned into corresponding sites in an adeno-associated viral backbone (pAAV_{CMV-HO-1}) containing the human cytomegalovirus (CMV) immediate early gene promoter and the bovine growth hormone polyadenylation signal flanked by the AAV inverted terminal repeats encoding the required replication and packaging signals. Packaging, propagation and purification of AAV viral particles was carried out using standard procedures.

Animals

Male Sprague Dawley rats (225-250 g) were purchased from Harlan Laboratories (Indianapolis, Ind.). The animals were maintained on a 12:12 hr light: dark cycle in at an ambient temperature of 24 degrees C. and 60% humidity. Food and water were provided ad libitum.

RNA Extraction and RT-PCR of hHO-1

For RT-PCR detection of hHO-1 transcripts, 100 ng of total RNA were used for first strand cDNA synthesis and PCR amplification with the One-Step Platinum Taq RT-PCR kit (Life Technologies). A 185 bp fragment was amplified for 30 cycles using the following primers: Forward, 5′GCTCTTTGAGGAGTTGCAGG-3′ (SEQ ID NO:1); Reverse, 5′-GTGTAAGGACCCATCGGAGA-3′ (SEQ ID NO:2)

Histology and Immunohistochemical Analysis

Hearts were flushed in situ with PBS (pH 7.4) and perfused retrogradely with 50 ml of 10% phosphate buffered formalin. The hearts were harvested, washed in PBS and post-fixed in 10% formalin overnight at 4° C. The specimens were processed for immunohistochemical analysis by standard methods. Sections were incubated in 1:400 dilution of rabbit anti-rat HO-1 polyclonal antibody (SPA 895, StressGen, Victoria, British Columbia. Canada) for 1 hr at room temperature and overnight at 4° C. and then incubated for 1 hr with 1:200 dilution of biotinylated goat anti-rabbit IgG embedded and sectioned at a thickness of 5 μm. Immunodetection was performed using the ABC method (Vectastain ABC kit, Vector Labs, Burlingame, Calif.). The sections were counterstained with 1% methyl green.

Western Blot Analysis

HO-1, apoptosis-related proteins Bax and Bcl-2, and pro-inflammatory proteins TNF-α, IL-1β and IL-6-associated immunoreactivities were detected in the total protein fraction prepared from left ventricle tissue homogenates by western blot. One hundred (HO-1) or fifty (Bax, Bcl-2, TNF-α, IL-6) micrograms of protein extract were electrophoresed, according to the method of Laemmli, on 10% SDS-polyacrylamide gels under reducing and denaturing conditions. The membranes were incubated in 1:1000 dilution of a human-reactive rabbit polyclonal anti-rat HO-1 antibody (SPA-895, StressGen) or in 1:100 dilution of rat-reactive rabbit polyclonal anti-human Bax (sc-19), Bcl-2 (sc-C21), TNF-α (sc-8301), IL-1β (sc-7884) and IL-6 (sc-7920) antibodies (Santa Cruz Biotechnologies, Santa Cruz, Calif.) for 2 hr. Immunodetection was performed with ECL (Amersham) after incubation in 1:3000 dilution of horseradish peroxidase anti-rabbit IgG for 1 hr. The membranes were exposed to Hyperfilm (Amersham) and the respective bands were quantified using NIH Image version 1.52 (National Institutes of Health, Bethesda, Md.).

### Cell Culture:

Human embryonic kidney cell line (HEK)-293 are maintained in complete growth medium, which consists of Eagle's minimum essential medium (MEM) supplemented with 1% nonessential amino acids and 10% fetal bovine serum (FBS). Cultures are incubated in a humidified atmosphere containing 5% C02 at 37° C. Coronary microvascular endothelial cells and primary rat cardiomyocytes are maintained in DMEM supplemented with 10% fetal calf serum (FCS) and specific growth medium (Clonetics, Walkersville, Md.).

### Transient Transfection:

Cells (4×10⁵) are plated into 6-cm glass petri dishes and grown for 16 to 18 hours until cell growth reach log phase and cell density is—50% confluent. Multiple sets of dishes in either duplicate or triplicate are prepared for each plasmid sample. Transfections are performed using 6 ul of lipofectamine (2 mg/ml) (Invitrogen, Carslbad, Calif.), 5 ug of plasmid containing the respective transgene constructs, and 0.5 ug of DNA containing the luciferase reporter gene (to correct for transfection efficiency) in 2 ml of serum-free MEM for 6 hours. Cells will then be harvested and assayed by the methods described below.

### Luciferase Assays.

Transfected cells are washed with PBS, removed from the glass surface using a rubber policeman and transferred into Eppendorf tubes. Cells are lysed using the “freeze-thaw” method, centrifuged, and the supernatants are collected. The supernatants are assayed for luciferase activity using a kit from Promega (Madison, Wis.). Briefly, an aliquot of supernatant are incubated with substrate at room temperature for 1 hour and the light intensity are measured using a Model2Oe luminometer (Berthold, Sunnyvale, Calif.). The amount of the transfected genes are normalized to the co-transfected luciferase activity to standardize the efficiency of transient transfections.

rAAV Production and Infection:

rAAV are produced in our Viral Core Facility by using the tHREeplasmid cotransfection system as described previously (Snyder et al, 1996). Briefly, HEK293 cells are grown in MEM containing 10% FBS. To generate AAV virus, the cells are cotransfected with 17 μg of transgene plasmid per dish along with 17 pg of plasmid pHLPI9 and 17 pg of plasmid pLadeno5 per dish. PHLP19 has AAV rep and cap genes, which provide the trans functions of rAAV adeno5 has the adenoviral VA, E2A and E4 regions that mediate rAAV replication. The medium are changed after 16 hours to complete MEM. After an additional 24 hours, the cells are collected and lysed by tHREe freeze-thaw cycles. Viral supernatants are generated by centrifugation at 10,000 g for 5 minutes and further purified by CsCI-gradient ultracentrifugation; the titer for each rAAV are determined by dot blot assay. This assay provides a titer of total number of particles per unit volume. The supernatant containing rAAV are stored in aliquots at −80 C and thawed for use immediately before each experiment.

### X-gal in Situ Staining.

Samples are fixed in 0.2% glutarahdehyde and 3% paraformaldehyde for 5 minutes, and washed twice with PBS. The samples are immersed in a staining solution containing 100 mM sodium phosphate (pH 7.3), 1.3 mM MgCl₂, 3 mM K₃Fe(CN)₆, 3 mM K₄Fe(CN)₆, and 5-bromo-4-chloro-3-indolyl-∃-D-galactoside (X-gal, I mg/ml) and incubated at 37° C. for 18 hours. The stained samples are washed twice with PBS and examined.

### Echocardiographic Determination of Left Ventricular Function:

Echocardiographic imaging of left ventricle dimensions is performed using a Hewlett Packard Sonos 5500 equipped with a 8-12 MHz vascular transducer. Measurements are performed at the mid-papillary level of the left ventricle in a blinded fashion. End diastolic diameter (EDD), end systolic diameter (ESD), anterior wall thickness (AWT) and posterior wall thickness (PWT) are obtained from the M-mode echocardiographic images according to the guidelines of the American Society for echocardiography leading-edge method. For each measurement, data from at least three consecutive cardiac cycles are averaged. End systolic (ESA) and end diastolic (EDA) are determined from the short axis view of the left ventricle at the papillary muscle level to evaluate LV ejection fraction (EF). Left ventricular fractional shortening (FS) and EF are calculated according to the following formulas: LV FS (%)=[(EDD−ESD)/EDD]×100; and LV EF (%)=[(EDA−ESA)/EDA]×100

### Histology and Immunohistochemical Analysis.

At 24 hr after reperfusion, hearts are flushed in situ with PBS (pH 7.4) and perfused retrograde with 50 ml of 10% phosphate buffered formalin. The hearts are harvested, washed in PBS and post-fixed in 10% formalin overnight at 4° C. The specimens are processed, embedded and sectioned at a thickness of 5 pm. Immunohistochemical staining are performed as described previously (Yet et al, 2001).

### Measurement of Heme Oxygenase Activity:

Total heme oxygenase is measured in the microsomal fraction isolated from left ventricular homogenates. Tissues are homogenized (˜3:1, ml:g tissue) in ice-cold homogenization buffer (30 mM Tris-HCl, pH 7.5), 0.25 M sucrose, 0.15 M NaCl) containing protease inhibitor cocktail (Sigma). The homogenates are centrifuged at 10,000 g for 15 minutes. The supernatant fraction is centrifuged at 100,000×g for 1 h. The microsomal pellet is resuspended in 50 mM potassium phosphate buffer (pH 7.4) and sonicated on ice for 5 seconds. Heme oxygenase activity is measured as the rate of appearance of bilirubin by a spectrophotometric method as previously described

### Assessment of Oxidative Stress and Oxidative Damage:

Oxidative damage is assessed by detecting oxidation-modified protein carbonyl groups in left ventricular homogenates using the OxyBlot kit (Intergen, New York, N.Y.) according to the instructions provided by the vendor, and by quantification of total lipid peroxides (malondialdehyde and 4-hydroxynoneal) using a commercially available kit (Calbiochem, Darmstadt, Germany). Immunostaining of formalin-fixed ventricular sections with polyclonal antibody MAL-2 (anti-malondialdehyde-lysine; donated by J. Witztum, La Jolla, Calif.) are used for in situ detection of oxidation-specific lipid-protein adducts as described previously (Melo et al, 2002). The integrated density of all bands corresponding to modified proteins in each lane was used for quantification of protein oxidation using a flatbed scanner and NIH Image 1.52 program.

### Determination of Apoptosis:

Apoptosis are determined by detection of internucleosomal fragmentation of genomic DNA using the Apoptotic DNA ladder kit (Roche, Indianapolis, Ind.), and by terminal deoxynucleotide transferase-mediated dUTP nick end-labeling (TUNEL) in paraffin-embedded sections, using the In Situ Cell Death detection anti fluorescein-dUTP peroxidase kit (Roche, Indianapolis, Ind.). For quantification of apoptosis by DNA laddering, genomic DNA are labeled with ³²P-dUTP (NEN, Cambridge, Mass.) using terminal deoxynucleotidyl transferase (Roche, Indianapolis, Ind.) for 1 hr at 37° C. The gel are exposed to Hyperfilm for 72 hr at −80° C. with intensifying screens. The integrated density of all the bands in the lane are used for quantification of apoptosis.

### Animal Surgery:

In preparation for surgery, the animals are lightly anesthetized initially by inhalation of 20% halothane:80 mineral oil mixture. Anesthesia are induced by intraperitoneal injection of a mixture of ketamine; xylazine (150:200 mg/kg BW) in sterile 0.9% NaCl and maintained with supplemental doses of the anasthetic mixture, as required. The animals are laid down in the supine position in an operating board and intubated with a blunt 17-gauge needle connected to a Harvard small rodent ventilator (Harvard Instruments, South Natick, Mass.). Tidal volume and ventilation rate are set at 2.5 ml and 60/mm, respectively during all open chest procedures. For continuous experiments, the animals are allowed to recover in their cage under a 100 W heat lamp for at least tHREe hours prior to being returned to the animal housing premises. The animals are monitored post-operatively for 24-48 hours and administered buprenorphine (0.2 mg/kg) at 18 hr intervals if deemed to be in distress. All animal experiments conforms with the guidelines for animal research from the National Institutes of Health, and all surgical and experimental procedures are approved by the Harvard Medical Area Standing Committee on Animals.

Statistical Analysis

All results are expressed as means±SE. One-way ANOVA coupled to Bonferroni multiple comparison test was used to compare differences between groups. P<0.05 was considered to indicate statistically significant difference.

### Example 2

### Gene Therapy for Long Term Protection from Oxidative Stress-Mediated Myocardiac Injury

Ischemia and oxidative stress are the leading mechanisms for tissue injury. The preventive/protective therapeutic methods described herein involve administration of a therapeutic gene with a vector that could confer long term transgene expression and consequently tissue protection from repeated ischemia/reperfusion injury. rAAV was used as a vector for direct delivery of the cytoprotective gene HO-1 into the rat myocardium. A single delivery of an AAV/HO-1 composition was found to reduce myocardial injury.

A construct containing a hHO-1 nucleic acid was introduced into normal rat hearts 8 weeks prior to coronary artery ligation and release. AAV-mediated transfer of hHO-1 gene led to a dramatic reduction (>75%) in left ventricular myocardial infarction induced by ischemia/reperfusion injury 8 later. The reduction in infarct size was accompanied by a decrease in myocardial lipid peroxidation, and in the level of expression of pro-apoptotic Bax and pro-inflammatory interleukin-1β proteins. An increase in antiapoptotic Bcl-2 protein level was also detected, suggesting that the transgene exerts its cardioprotective effects by reducing oxidative stress and associated inflammation and apoptotic cell death. These data documents the beneficial therapeutic effect of direct rAAV-mediated transfer of a cytoprotective gene in long-term myocardial protection from ischemia/reperfusion injury. Experimental results further indicate that this approach leads to sustained tissue protection from repeated episodes of injury.

Intramyocardial Gene Delivery

For direct gene delivery into the myocardium, a small oblique thoracotomy was performed lateral to the midsternal line, in the third intercostal space to expose the heart. A total of 4×10¹¹ particles of rAAV-hHO-1 vector in a final volume of 750 μl were delivered subepicardially with a curved 25 gauge needle (supplier) into five sites along the anterior and posterior left ventricular wall. Control animals received an equivalent volume of either sterile Ringers saline or rAAV vector expressing the LacZ reporter gene. The area of injection corresponded to the region of the myocardium supplied by the left ascending coronary artery (LAD), as previously determined in multiple separate experiments using Evans blue dye exclusion after LAD ligation. After injection, the exposed heart was monitored for 2-5 minutes for resumption of normal sinus rhythm. The chest incision was then closed in layers with 3.0 suture (Ethicon) and the animals were allowed to recover. Mortality rate during and post-surgery was less than 1% in all groups.

Acute Ischemia-Reperfusion Model

A midsternal thoracotomy was performed to expose the anterior surface of the heart. The proximal LAD was identified and a 6.0 suture (Ethicon) was placed around the artery and surrounding myocardium. Regional left ventricular ischemia was induced for 30 min by ligation of LAD. Ischemia was confirmed by discoloration of myocardium and by changes in cardiac rhythm. Sham-operated animals served as surgical controls and were subjected to the same surgical procedures as the experimental animals, with the exception that the LAD was not ligated. At the end of the ischemia period, the ligature was loosened and left in place. Reperfusion was confirmed by hyperemia and resumption of normal rhythm. The incision was closed and the animals were allowed to recover.

Morphometric Determination of Infarct Size

Twenty four hours after reperfusion, the LAD was re-ligated and 0.3-0.4 ml of 1% Evans Blue in PBS (pH 7.4) were retrogradely injected into the heart via the catheter to delineate the non-ischemic area. The heart was excised and rinsed in ice cold PBS. Atrial tissue and large vessels were removed and 5-6 biventricular sections of similar thickness were made perpendicular to the long axis of the heart. The sections were incubated in 1% triphenyl tetrazolium chloride (TTC, Sigma Chemicals) in PBS (pH 7.4) for 15 min at 37° C. and photographed on both sides. The slides were projected at approximately 10× magnification and traced on Quad 10 to 1″ graph paper. Area at risk and infarct area were delineated and calculated for both sides of the section. The cumulative areas for all sections for each heart were used for comparisons. Infarct size was expressed as the ratio of infarct area to area at risk.

Intramyocardial Delivery of a HO Gene Using Adeno-Associated Virus

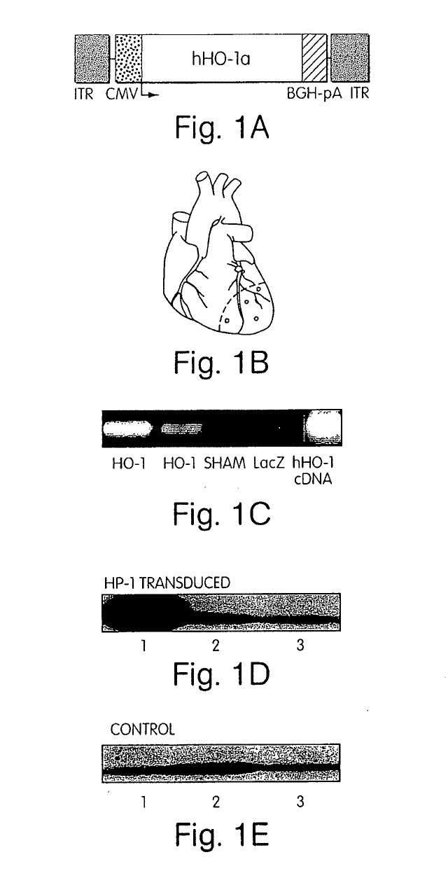

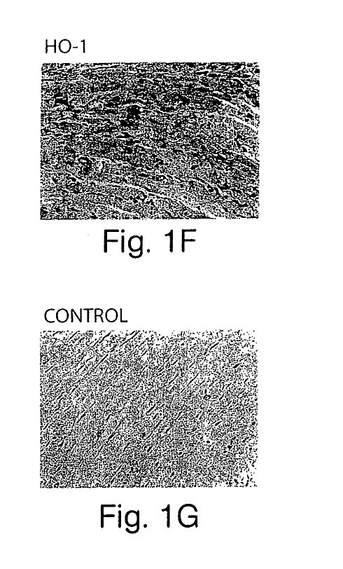

FIG. 1A is a schematic diagram of rAAV_{CMV-hHO-1} viral vector expressing hHO-1 cDNA under transcriptional control of the human cytomegalovirus (CMV) early gene promoter. ITR indicates the AAV inverted terminal repeats encoding replication and packaging signals. BGH-pA indicates the bovine growth hormone polyadenylation signal. FIG. 1B shows the strategy for rAAV-mediated intramyocardial delivery of the hHO-1 gene. The rAAVCMV-hHO-1 vector (FIG. 1A) was injected at five sites corresponding to the area at risk after ligation of the LAD (FIG. 1B). FIG. 1B shows the approximate area at risk of infarction (delineated by dotted line, based on Evans Blue exclusion) after ligation of left anterior descending coronary artery and sites of injection (filled circles) of vector. FIG. 1C shows the results of RT-PCR detection of hHO-1 transcripts in left ventricle tissue 8 weeks after rAAV-mediated intramyocardial gene transfer, and FIGS. 1D-E show the results of a Western blotting analysis of distribution and abundance of HO-1 protein with human reactive anti-rat HO-1 antibody. FIGS. 1F-G are photomicrographs showing the localization of HO-1 protein in paraffin-embedded cross sections. Staining corresponding to HO-1 immunoreactivity was detected in sections from rAAV_{CMV-hHO-1} transduced hearts, but was absent in untransduced hearts. Tissue sections shown in FIGS. 1F-G were counterstained with methyl green and viewed at a magnification of 400×.

The injection approach reproducibly results in 40-60% transduction of the targeted area as evaluated by rAAVCMV-LacZ vector. Eight weeks after transduction, transgene mRNA was detected in hHO-1-transduced hearts, but not in LacZ-transduced or untransduced (sham) hearts (FIGS. 1C-E). Immunoreactive HO-1 protein detected with a human reactive anti rat HO-1 antibody was highest in the apical region in hHO-1 transduced hearts (FIGS. 1D, E), corresponding to the area targeted by gene transfer. Immunohistochemical detection of HO-1 protein in paraffin-embedded apical ventricular cross sections from hHO-1-transduced hearts revealed intense staining corresponding to HO-1 immunoreactivity (FIG. 1F), not seen in equivalent sections prepared from untransduced (control) hearts (FIG. 1G).

Effect of rAAV-Mediated hHO-1 Gene Transfer on Left Ventricular Infarct Size

Eight weeks after gene transfer, the animals were subjected to 30 minutes of regional left ventricular ischemia by ligation of the proximal left anterior descending coronary artery. Gross histological analysis of TTC-stained biventricular sections prepared at 24 hr after reperfusion revealed attenuated myocardial injury in hHO-1-transduced hearts compared to widespread necrosis in the rAAV-Lacz and saline-injected controls. Planar morphometric analysis of area at risk (AAR) was carried out and the values expressed as means±SE. AAR did not differ significantly between groups, but infarct area and infarct size were significantly reduced in HO-1-treated hearts compared to LacZ-transduced and untransduced hearts (*, P<0.05). Sham-operated animals (n=6) did not show evidence of ischemia or infarction.

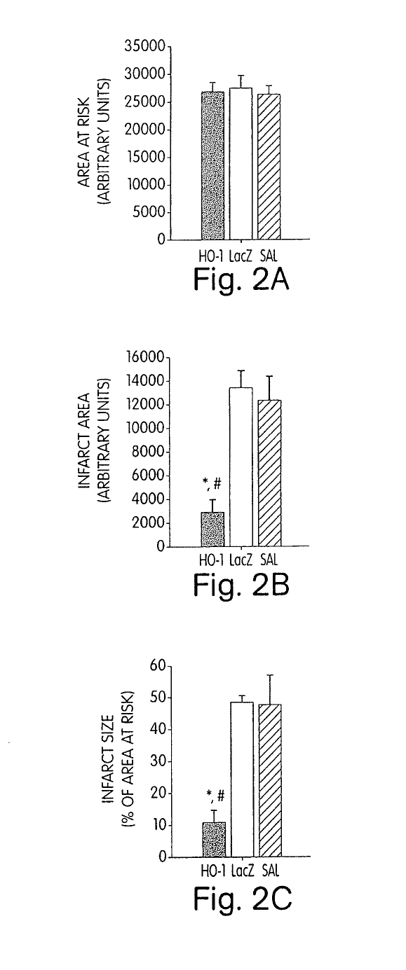

Planar morphometric analysis of serial cross sections showed that despite comparable areas at risk (FIG. 2A) as evaluated by Evans blue staining after ligation, the infarct area (FIG. 2B) and infarct size (% of area at risk) (FIG. 2C) were significantly smaller in the hHO-1-transduced hearts than in control hearts (HO-1, 11%; Lac-Z, 49; Saline 48%), corresponding to 75%-80% relative reduction in infarct size in the HO-1-transduced hearts.

HO Enzyme Activity and Protein Expression in Whole Left Ventricle Homogenates after 30 min of Ischemia and 24 hr of Reperfusion

Total basal heme oxygenase activity was determined in left ventricle microsomal extracts from hHO-1-transduced (n=6), LacZ-transduced (n=5), saline-injected (n=7) and sham-operated (n=5) animals. Values are means±SE.

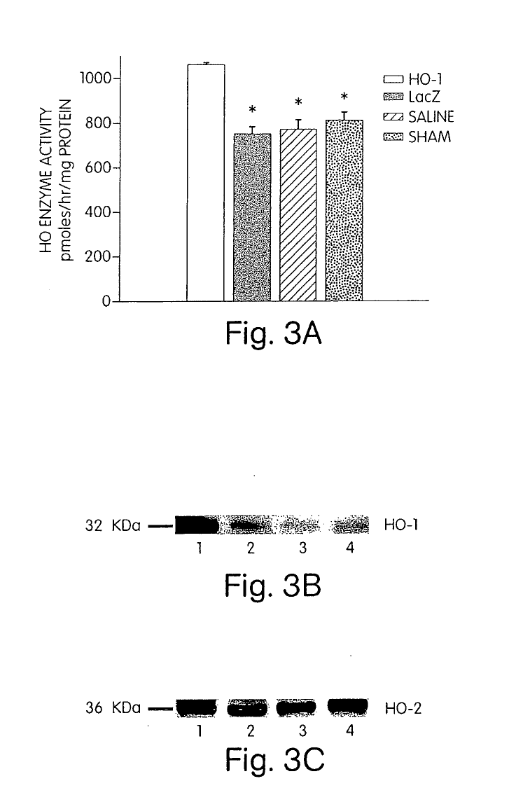

Basal microsomal heme oxygenase activity in whole homogenates prepared from left ventricle 24 hr after reperfusion was significantly elevated (≅35%-40%) in the hHO-1-transduced hearts relative to controls (FIG. 3A), coincident with a comparable % increase in HO-1 protein abundance (FIGS. 3B,C). No differences in HO-1 protein abundance or enzyme activity were found between controls and sham-operated animals, indicating that potential I/R-mediated induction of endogenous heme oxygenase activity had subsided at 24 hrs after reperfusion. Furthermore, heme oxygenase 2 (HO-2) protein abundance did not differ between groups (FIG. 3C), indicating that the relative difference in heme oxygenase activity between hHO-1-transduced hearts and controls is due to overexpression of the transgene.

Effect of rAAV-Mediated hHO-1 Gene Transfer on Oxidative Stress-Induced Lipid Peroxidation and Expression of Apoptosis-Related Proteins and Inflammatory Cytokines after 30 min of Ischemia and 24 hr of Reperfusion

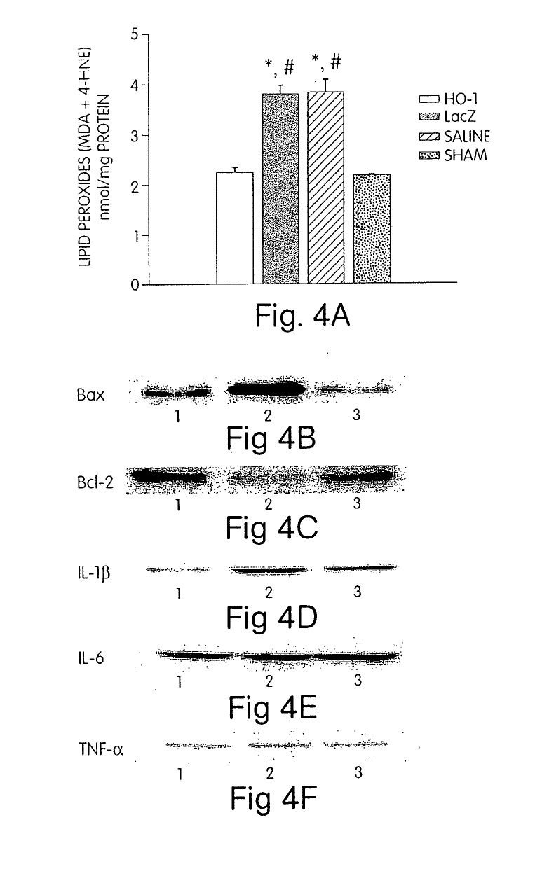

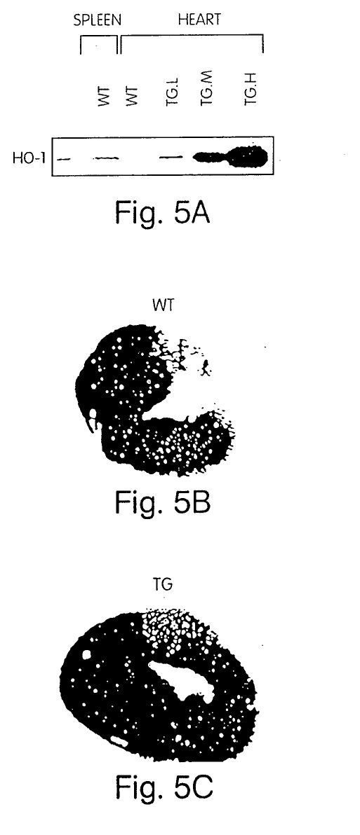

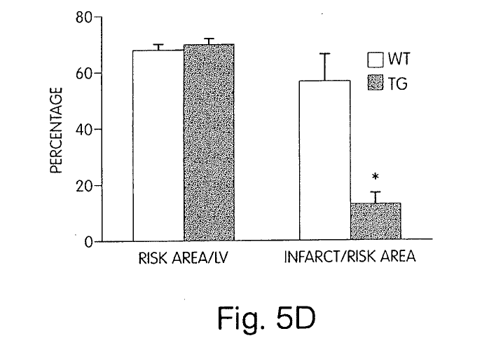

Total lipid peroxides (malondialdehyde, MDA and 4-hydroxy-(E)-nonenal, 4-HNE) were measured in HO-1-transduced (n=8), LacZ-transduced (n=5), saline injected (n=8) and sham (n=6) operated animals. The effect of rAAV-mediated hHO-1 gene transfer on apoptosis-related proteins an inflammatory cytokine expression was measured in left ventricle homogenates after 30 min of ischemia and 24 hr of reperfusion. At 24 hr after reperfusion, total lipid peroxide (malondialdehyde and 4-hydroxy-(E)-nonenal) concentration was significantly elevated in the LacZ transduced and saline injected hearts compared to the hHO-1-transduced and sham-operated hearts (FIG. 4A). Lipid peroxide accumulation in the hHO-1-transduced hearts was comparable to the levels in sham treated hearts, indicating that hHO-1 gene transfer reduces I/R induced lipid peroxidation and oxidative myocardial damage. Concomitant with the reduction in oxidative stress, we found a relative increase in anti-apoptotic Bcl-2 protein levels and lower abundance of pro-apoptotic Bax protein and pro-inflammatory cytokines IL-1β in left ventricle from hHO-1-transduced hearts compared to LacZ and saline-treated controls (FIGS. 4B-F). The levels of other cytokines (IL-6, TNF-α) and apoptosis-related proteins (Bad, Bcl-XL, p53) did not differ significantly between groups.

HO-1 Gene Therapy for Long Term Cardioprotection and Treatment of Chronic Cardiac Disorders

Myocardial infarction resulting from ischemic coronary heart disease is the leading cause of death in the Western world. The current findings show that rAAV-mediated intramyocardial delivery of the cytoprotective gene HO-1 into normal hearts 8 weeks prior to injury confers sustained myocardial protection from ischemia/reperfusion injury. These data indicate that (1) rAAV is an efficacious vector for gene delivery with long-term, stable transgene expression in the myocardium and that (2) HO-1 is an effective therapeutic gene for myocardial protection form ischemia/reperfusion-induced injury. The compositions are useful for long-term preventive and/or adjunct cardioprotective therapy in humans.

Several biological properties of rAAV and HO-1 render them a suitable combination for long-term cardioprotective gene therapy. First, rAAV is non-immunogenic, thereby circumventing activation of the host immune response. Secondly, rAAV has the ability to integrate into the host genome, thus, providing stable expression of the transgene and, potentially, indefinite production of the therapeutic protein. HO-1 neutralizes the potent pro-oxidant activity of heme and that its multiple catalytic by-products bilirubin, carbon monoxide (CO) and free iron together exert powerful, pleiotropic cytoprotective effects. Bilirubin is a potent endogenous antioxidant that scavenges peroxyl radicals and reduces peroxidation of membrane lipids and proteins. CO is a vasodilator and powerful anti-inflammatory and antiapoptotic agent. Free iron stimulates the synthesis of the iron binding protein ferritin, which reduces iron-mediated formation of free radicals and upregulates several key cytoprotective genes. The current findings show that HO-1 gene transfer exerts significant and sustained cardioprotection, despite a relatively modest increase in basal enzyme activity.

In concordance with the cytoprotective actions of HO-1, the data indicate that the attenuated response of hHO-1-transduced hearts to I/R-induced myocardial injury is due to reduction in myocardial oxidative stress and in apoptotic and inflammatory activities. The levels of myocardial lipid peroxides accumulation, a common marker of oxidative stress, and IL-1β, and Bax proteins are decreased by hHO-1 gene transfer in the ischemia/reperfused myocardium in parallel with an increase in antiapoptotic Bcl-2 protein, indicating that HO-1 confers cardioprotection by exerting anti-oxidant, antiapoptotic and anti-inflammatory properties. The protective effects of HO-1 may be exerted by separate or cumulative events resulting from activation of single or multiple cytoprotective cascades.

The data described above demonstrates that rAAV-mediated intramyocardial delivery of hHO-1 results in prolonged transgene expression and, consequently long-term myocardial protection as shown by a dramatic reduction in infarct size induced by ischemia/reperfusion. This result is surprising because the therapeutic gene was introduced many weeks prior to injury. These findings demonstrate for the first time the therapeutic potential of rAAV vector/HO-1 gene combination for long-term protection from myocardial ischemia/reperfusion injury. Given the prevalence of coronary heart disease, such a combination of vector and therapeutic gene is a useful strategy for long-term cardioprotective gene therapy for chronic cardiac conditions in humans.

### Effect of HO-1 Gene Transfer on Post-Infarct Ventricular Hemodynamics

The effect of hHO-1 gene transfer on left ventricular functional recovery following myocardial infarction using two dimensional echocardiographic imaging and analysis at the mid-papillary level was assessed. Pre and post-infarction echocardiographic measurements were obtained at baseline just prior to ligation and at 2 weeks after reperfusion (n=4-6 group). The 2 weeks post infarction interval is generally accompanied by accentuated remodeling and changes in ventricular dimensions. The data demonstrates that post infarction fractional shortening (FS) and ejection fraction (EF) did not differ significantly from basal values in the hHO-1 treated groups, but was significantly reduced in the LacZ and saline treated groups (Table II).

TABLE II
Effect of HO-1 gene transfer on post-Infarct ventricular hemodynamics by 2-D
echocardiography

| | SALINE | | LacZ | | HO-1 | |
|---|---|---|---|---|---|---|
| PARAMETER | Pre | Post | Pre | Post | Pre | Post |
| IVS (mm) | .157 ± .0038 | .157 ± .0040 | .156 ± .0049 | .171 ± .0073 | .143 ± .0086 | .145 ± .0106 |
| PW (mm) | .176 ± .0069 | .160 ± .0062 | .176 ± .0069 | .160 ± .0062 | .170 ± .0088 | .162 ± .0146 |
| LVDD (mm) | .831 ± .0326 | .990 ± .0069* | .872 ± .0259 | .970 ± .0082* | .873 ± .0267 | .911 ± .0350 |
| LVSD (mm) | .550 ± .0345 | .820 ± .0233* | .541 ± .0261 | .734 ± .0174* | .577 ± 0.031 | .600 ± 0360 |
| FS (%) | 34.1 ± 1.7 | 17.2 ± 2.0* | 38.1 ± 2.4 | 24.3 ± 1.7* | 33.9 ± 2.1 | 34.2 ± 1.6 |
| EF (%) | 71.2 ± 2.2 | 43.0 ± 3.9* | 76.0 ± 2.7 | 56.5 ± 3.0* | 70.9 ± 2.7 | 71.4 ± 2.0 |

*P < 0.05 Pre vs Post.

Similarly no significant post infarction changes in ventricular chamber dimensions (left ventricular diastolic diameter, LVDD, left ventricular systolic diameter, LVSD) or wall dimensions (posterior wall, PW, intraventricular septum, IVS) were observed in the HO-1 treated animals. In contrast there was a significant increase in post-infarct ventricular dimensions in both the LacZ and saline treated groups. Consistent with the echocardiographic data, we failed to observe histopathological evidence of myocardial damage in tissues collected from the HO-1 treated animals at termination of the experiments (around 13 weeks post transduction), whereas significant scarring was present in the control animals. This data indicate that HO-1 gene transfer heads to marked myocardial protection and healing which is accompanied by improved post-reperfusion functional recovery relative to untreated animals.

Myocardial Protection Using AAV Delivery of ecSOD Gene

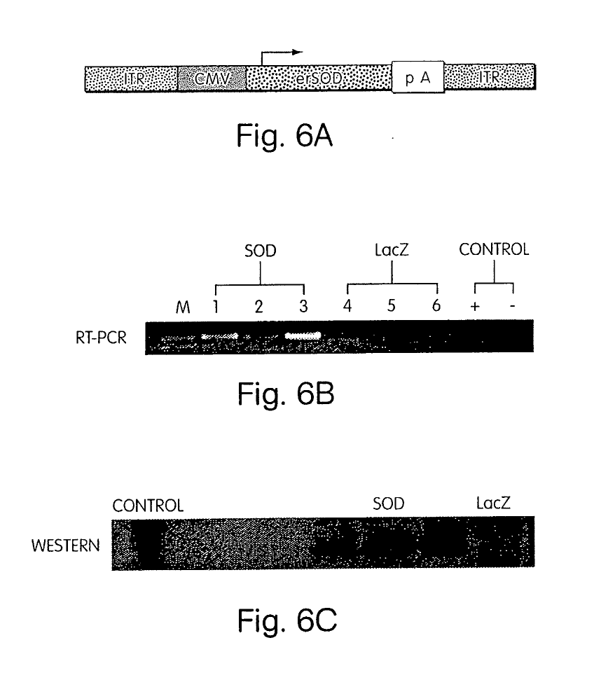

The delivery of human ecSOD in protection from I/R injury was tested. The vector consisted of an AAV backbone (pAAV-CMV-ecSOD) containing the human cytomegalovirus (CMV) immediate early gene promoter and the polyadenylation signal of the growth hormone flanked by AAV inverted terminal repeats. An AAV construct containing the open reading frame of ecSOD cloned downstream of the CMV promoter to allow constitutive expression (FIG. 6A).

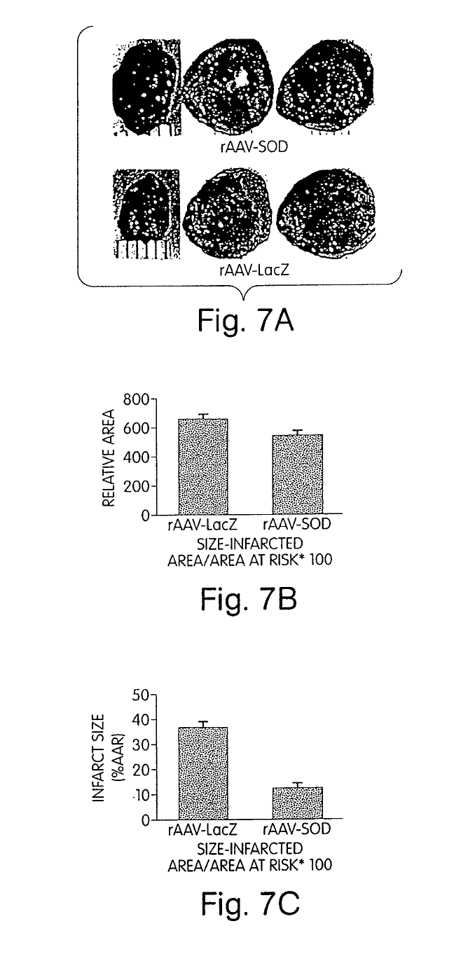

This AAV vector was injected into rat hearts as described above. Eight weeks after transduction, transgene mRNA (FIG. 6B) and immunoreactive human ecSOD protein (FIG. 6C) were detected in transduced hearts. Acute myocardial infarction by LAD ligation for 30 minutes followed by reperfusion for 24 hours eight weeks after gene transfer to determine whether rAAV mediated intramyocardial delivery of human ecSOD to the risk area could confer long term protection from I/R injury. These findings are summarized in FIG. 7. Gross histological analysis of TTC-stained biventricular sections prepared at 24 hours after reperfusion showed that animals transduced with the human ecSOD gene showed attenuated myocardial injury (FIG. 7A) compared to widespread necrosis in the rAAV-lac Z transduced animals. Planar morphometric analysis of serial sections showed that despite comparable areas at risk (FIG. 7B), the infarct size (% of area at risk) (FIG. 7C) was dramatically reduced (>75%) in the rAAV-ecSOD-treated animals compared to the control animals receiving saline or the biologically silent LacZ reporter gene. In addition, echocardiographic studies performed 2 weeks later showed that rAAV-ecSOD significantly attenuated the I/R induced impairment in left ventricular function as assessed by fractional shortening

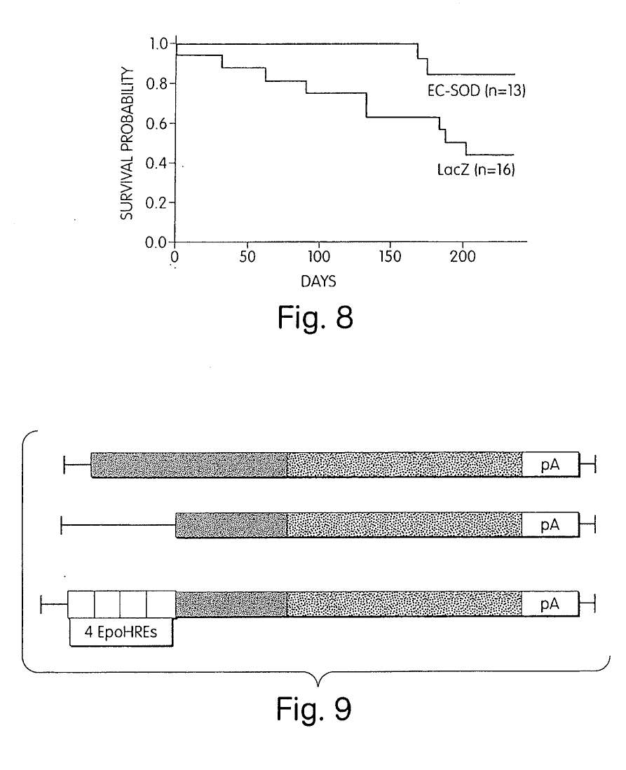

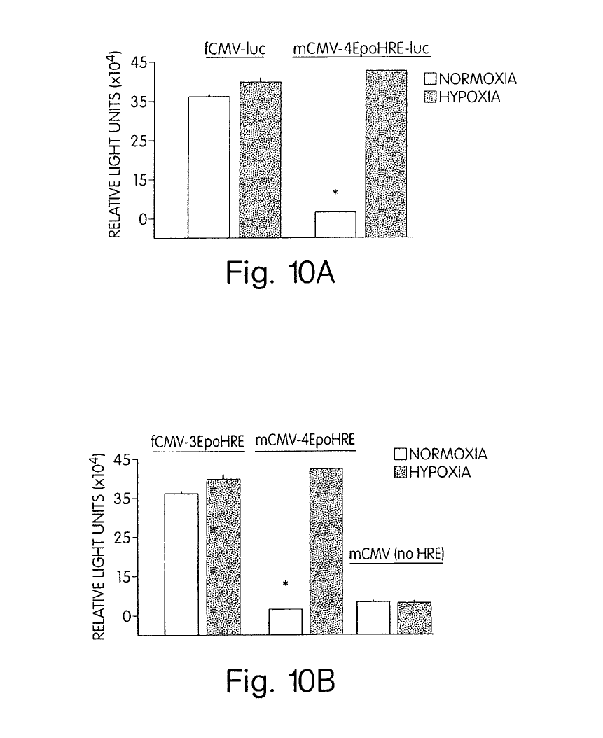

A subset of animals were injected with the vectors, underwent the h/R injury and studied for survival. Animal survival was assessed for a year, post the ischemia/reperfusion injury (FIG. 8). To date, animals injected with the AAV-ecSOD showed significant improvement in survival 13) when compared to animals treated with AAV-lacZ alone. As shown in FIG. 10, 9/13 animals in the AAV-hEC-SOD group LacZ(n.16) survived the injury while in the AAV-lacZ group only 3/16 survived.

Duration and Tissue Distribution of rAAV-LacZ Expression Following Intra-Myocardial Gene Transfer.

AAV based vectors are increasingly being used as viable vectors for cardiovascular gene therapy due to their low toxicity, high transduction efficiency and low immunogenicity. However, its high tropism may result in widespread tissue distribution despite local gene delivery. Previous studies have investigated the distribution of AAV following injection into organs such as brain and liver; however little is known regarding the distribution of the virus in non-target organs following intramyocardial administration and the potential side effects associated with such distribution. This study was designed to investigate the duration and distribution of AAV-LacZ following intramyocardial gene transfer. Six week-old Lewis rats were injected with rAAV-lacZ at five sites in the anterior wall of the left ventricle. Another group of uninjected rats were used as control. Fifteen months after viral injections, animals were fixed with 3% paraformaldehyde, tissues were removed and stained for 3-gal activity in PBS at pH=8.5 to distinguish between the endogenous and bacterial a-gal activity. When compared with the control group, we observed positive ˜3-gal activity in the majority of the tissues (heart, liver, and testis) even after 15 months after intramyocardial injection except in lungs and aorta. The most surprising result was that intramyocardial gene transfer led to gene expression in the gonads. These preliminary results indicate that spillover occurs into other tissues.

### Example 3

### Regulatable Gene Expression Using Hypoxic Response Element Constructs In Vitro

The data has demonstrated the efficacy of the intramyocardial HO-1 or ecSOD delivery via AAV vector in protecting against I/R injury. In order to develop regulatable expression of the therapeutic gene induced by specific pathophysiological stimuli several hypoxia inducible vectors were constructed and tested the efficiency of these vectors to induce gene expression during in vitro hypoxia (FIG. 9). These vectors contain multiple tandem repeats of hypoxia responsive elements from the erythropoietin gene (Epo HREs), which were placed upstream of minimal CMV promoter followed by the luciferase gene. In addition, control vectors containing full length and minimal CMV promoter alone were constructed.

To test the efficacy of the hypoxia response elements to induce hypoxia mediated gene expression, HEK 293 cells were transfected with the following vectors: pGL3-4EpoHRE-mCMV-luc, pGL3-mCMV-luc and pGL3-fCMV-luc. A shown in FIG. 10A under basal conditions, cells transfected with the pGL3-fCMV vector exhibited a 10 fold higher level of expression as measured by luciferase activity when compared to cells transfected with vector containing mCMV promoter. However under hypoxic conditions, cells transfected with pGL3-mCMV-4Epo-HRE showed a 10 fold greater induction in luciferase activity as measured by relative light units (RLUs). In contrast, neither the pGL3-fCMV nor the vector containing mCMV alone responded to hypoxia (FIG. 10B).

These results indicate that pGL3-4EpoHRE construct containing a minimal CMV promoter results in low basal levels of gene expression which is then induced 10 fold under hypoxic conditions. On the contrary a vector containing just the mCMV promoter without HREs gave a very low basal and did not result in the induction of luciferase activity (FIG. 10B). Since the eventual goal of this project is to test the viability of the hypoxia inducible AAV vectors to confer in vivo myocardial protection, we have also constructed the AAV vectors with up to five tandem repeats of hypoxia response elements from the erythropoietin gene, the minimal CMV promoter and GFP as the reporter gene and tested their efficiency to induce GFP expression under hypoxia The preliminary result showed that 5xEpoHRE resulted in further increased GFP expression in response to hypoxia.

### Example 4

### Long-Term Effect of Intramyocardial Ho-1 Gene Delivery on Post-Infarction Myocardial Histopathology

In agreement with echocardiographic evidence of functional recovery and normalization of ventricular dimensions after infarction in the rAAV-HO-treated animals this data shows the absence of histopathological evidence of myocardial damage in the HO-1 treated animals one month after infarction. Since fibrosis and chamber remodeling in rats are usually resolved at four weeks after infarction, a one month post-infarction was chosen as the time point for evaluation of the long-term effect of intramyocardial HO-1 gene delivery on post-infarction myocardial histopathology. Gross histological examination of TTC-stained biventricular sections showed a dramatic reduction in infarct size in the rAAV-HO-1-treated compared to the LacZ-treated animals.

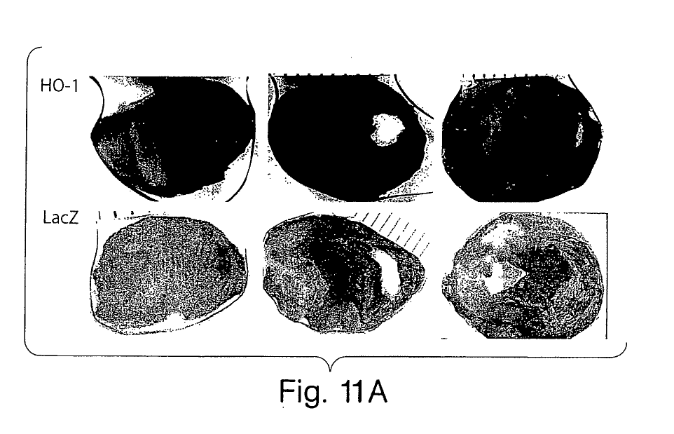

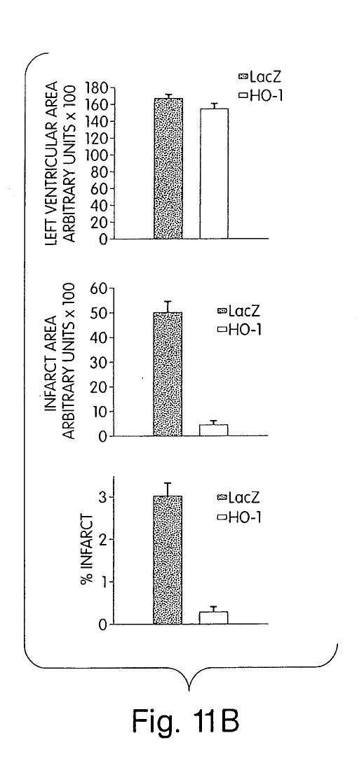

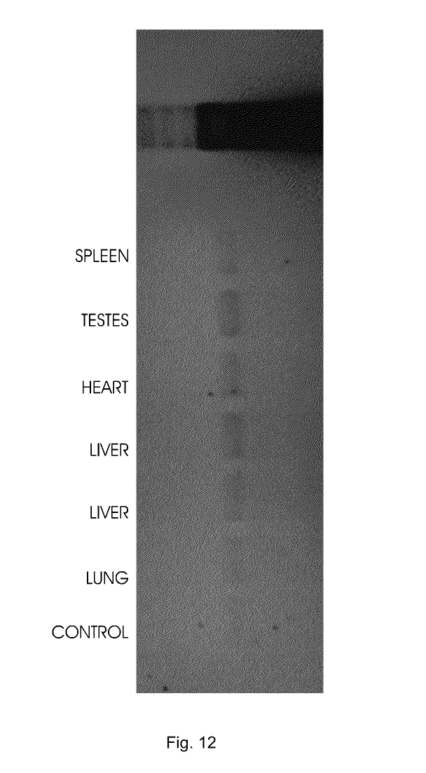

Planar morphometric analysis of the sections showed approximately 90% reduction in infarct size in the HO-1 treated relative to the LacZ control animals (FIG. 11B). In concordance with the smaller infarcts, there was little histological evidence of myocardial injury in the HO-1 treated animals. H&E and Masson trichrome staining of paraffin sections from the infarcted area showed widespread scarring and fibrosis in the LacZ-control animals, whereas no evidence of injury was seen the HO-1 treated animals (FIG. 2). Extensive inflammatory cell infiltration (CD45 positive cells) was seen in the infarcted region of the control animals, but not in HO-1 treated animals. In conjunction with the echocardiographic assessment of left ventricular function and chamber dimensions, these data indicate that AAV-mediated HO-1 gene transfer confers sustained myocardial protection that is accompanied by functional recovery and prevention of myocardial injury and remodeling after acute myocardial infarction.

This establishes the ability of rAAV-mediated intramyocardial delivery of cytoprotective genes such as HO-1 and ec-SOD as a therapeutic strategy for long-term myocardial protection.

### Example 5

### Evaluation of the Safety of Intramyocardial Delivery of Cytoprotective Genes

The long-term expression of the bacterial β-galactosidase reporter gene in various remote organs after intramyocardial delivery by AAV, in order to evaluate the safety of this delivery method. The rationale for choosing this delivery route was to restrict the expression of the therapeutic protein to the required sites, thereby minimizing the occurrence of potentially undesirable side effects resulting from expression of the protein in remote organs. However, there have been reports indicating ectopic transgene following intramuscular delivery. Although the pathophysiological consequences of spillover would in many cases be negligible, situations may also arise where ectopic expression of the therapeutic transgene may have pathophysiological implications. Furthermore, the potential risk of germ cell line transmission of the transgene would carry serious ethical implications, particularly in the context of human gene therapy.

Similar to previous observations with intramuscular injection, these results show molecular and histological evidence of long-term ectopic reporter transgene expression in various extracardiac tissues following intramyocardial delivery with a constitutive rAAV vector. Expression of LacZ transcripts in the liver, kidneys and testis 6 months after intramyocardial delivery of 4×10¹¹ rAAV particles was detected (FIG. 11). A significant number of LacZ-positive cells were also found in frozen sections prepared from these organs. These findings indicate that local administration of the vector to highly vascularized tissues such as the heart and skeletal muscle can lead to significant systemic spillover and expression of the transgene at remote locations. The use of AAV vectors incorporating tissue-specific cis-acting promoter elements however may restrict expression of the therapeutic protein to the specific tissues or cell types involved in the pathophysiological process. Such a strategy would preclude the manifestation of possible side effects associated with ectopic expression of the therapeutic transgene, thus enhancing the safety of the therapy.

Other embodiments are within the following claims.

What is claimed is:

1. A composition comprising a nucleic acid encoding a human hemeoxygenase-1 polypeptide operably linked to a constitutive promoter.

2. The composition of claim 1, wherein the nucleic acid is a vector selected from the group consisting of an adeno-associated virus vector, a lentivirus vector and a retrovirus vector.

3. The composition of claim 1, wherein the nucleic acid is an adeno-associated virus vector.

4. The composition of claim 1, wherein the promoter is a human cytomegalovirus immediate early promoter.

5. The composition of claim 1, wherein the nucleic acid further comprises a bovine growth hormone polyadenylation signal.

6. The composition of claim 5, wherein said bovine growth hormone polyadenylation signal is flanked by adeno-associated viral inverted terminal repeats.

7. The composition of claim 5, wherein the nucleic acid is an adeno-associated virus vector, and the promoter is a human cytomegalovirus immediate early promoter.

8. The composition of claim 7, wherein the nucleic acid further comprises a bovine growth hormone polyadenylation signal.

9. The composition of claim 8, wherein said bovine growth hormone polyadenylation signal is flanked by adeno-associated viral inverted terminal repeats.

10. A method of preventing an ischemic or reperfusion related injury in a subject, comprising administering to a subject at risk of suffering an ischemic or reperfusion-related injury a composition according to any one of claim 68-76.

11. The method of claim 10 in which the subject is suffering from a chronic cardiac disorder.


SEQUENCE LISTING (2 sequences)

SEQ ID NO: 1 - DNA, 20 bp, Artificial sequence
  Chemically synthesized primer
gctctttgaggagttgcagg

SEQ ID NO: 2 - DNA, 20 bp, Artificial sequence
  Chemically synthesized primer
gtgtaaggacccatcggaga